(19) United States
(12) Patent Application Publication
(10) Pub. No.: US 2006/0149094 A1
(43) Pub. Date: Jul. 6, 2006

(54) Process for generating pivaloylacetate esters via carbonylation of chloropinacolone

(76) Inventor: Joseph Robert Zoeller, Kingsport, TN (US)

Correspondence Address: Michael J. Blake;Eastman Chemical Company, P.O. Box 511, Kingsport, TN, 37662-5075, US

(21) Appl. No.: 11/027,238

(22) Filed: Dec. 30, 2004

(51) Int. Cl. C07C 67/36

(52) U.S. Cl. 560/175

(57) ABSTRACT

Pivaloylacetate esters (1) are a useful intermediates for producing chemicals for use in photographic and xerographic reproduction processes. Such esters can be generated by contacting chloropinacolone (2) with CO, an alcohol of formula R¹OH, and a base in the presence of a catalyst comprising a source of palladium and certain coordinating trisubstituted phosphines of formula (R³)₃P.

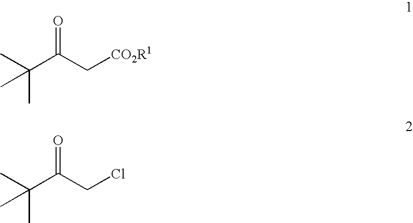

### FIELD OF THE INVENTION

The present invention relates to a novel and efficient process for generating pivaloylacetate esters by contacting chloropinacolone with carbon monoxide and an alcohol in the presence of a base and a palladium catalyst.

### BACKGROUND

Pivaloylacetate esters (4,4-dimethyl-3-oxo-1-pentanoate esters, 1 below) are useful intermediates in the production of substances used in photographic and xerographic reproduction processes. These esters have been synthesized by several means in the past, including a classical Claisen condensation process in which an acetate is condensed with a pivalate ester. (See, Eyer, U.S. Pat. No. 5,144,057 (1992); Rathke et al., Tetrahedron Letters, 2953 (1971).) Also used is a condensation of pinacolone (3,3-dimethyl-2-butanone) with a carbonate ester. (See, Sun et al., Huanong Shikan, 15, 33 (2001); Boaz et al. U.S. Pat. No. 6,143,935 (2001); Harada et al. (Jpn. Kokai Tokkyo Koho) JP 09110793 (A2) (1997); Harada et al., JP 07215915 (1995); Harada et al., JP 06279363 (1994); Harada et al., JP 06279362 (1994); Iwasaki et al., JP 3371009 (2003) (B2); Renner et al., U.S. Pat. No. 4,031,130 (1977); Renner et al., GB 1,491,606 (1977).) An inherent feature of these condensations is that they require greater than one equivalent of an expensive strong base, such as sodium hydride or a sodium alkanoate, which can not be recovered. In addition, although higher yields may help compensate for the expense, carbonate esters are more expensive than acetates and yields are still only moderate.

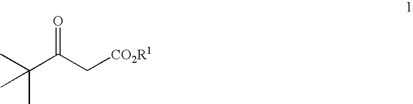

An alternative method involves acyl exchange between pivaloyl chloride and an acetoacetate ester. (See, Shinya et al., U.S. Pat. No. 6,570,035 (2003); Sato et al., JP 10025269 (1998)(A2); Sato et al, JP 2000143590 (2000) (A2); Suenobe et al., JP 63057416 (1988); Mainzer et al., DD 235636 (1986); Yoshitomi Pharmaceutical Industries, JP 57070837 (1982); Rathke et. al., J. Org. Chem., 50, 2622 (1985).) This process reacts an alkaline earth base, such as magnesium, with an acetoacetate ester and pivaloyl chloride. These have normally required an equivalent of base or more, but a process using a catalytic amount of magnesium base has been recently developed. (See, Yamada et. al. U.S. Pat. No. 6,570,035 B2 (2003).) These processes use moderately expensive acetoacetates and give only moderate yields but are competitive with the condensation processes described above. Earlier processes using malonates instead of acetoacetates are disfavored because of the even higher expense of the malonate intermediates and much lower yields as compared to the aforementioned Claisen condensations or acyl exchange processes. (See, Sheldon et al. U.S. Pat. No. 4,656,309 (1987); Vlassa et al., J. fur Praktische Chemie (Liepzig), 322, 821 (1980).)

Other routes to pivaloylacetate esters include the condensation of pinacolone with an oxalate ester followed by a pyrolysis (decarbonylation) to generate the desired pivaloylacetoacetate ester (Yang et al., Jingxi Huagong, 15,49 (1998); Mitaru et al., JP 08027065 (1996); Mizutare et al., U.S. Pat. No. 5,679,830 (1997); Werner et al., U.S. Pat. No. 4,661,621 (1987)), and the palladium catalyzed coupling of pivaloyl chloride with BrZnCO₂R. (Sato et al., Chemistry Letters, 1559 (1982).) Drawbacks to such processes include the need for expensive reagents and the processes generate even more waste than earlier processes. Thus, there still exists a need for a more efficient (e.g., lower waste, lower cost) method to generate pivaloyl esters.

Some have recently reported the carbonylation of chloroacetone and a-chloroacetophenone derivatives using triphenylphosphine palladium catalysts to generate the aceotacetate and benzoylacetate esters. (Lapidus et al., Russian Chemical Bulletin, Int. Edit., 50, 2239 (2001); Lapidus et al., Synthesis, 317 (2002).) These processes only demonstrate a maximum of 100 catalyst turnovers (defined as moles of pivaloylacetate ester product/mol of Pd added), despite using very expensive palladium catalysts. By contrast, viable processes using palladium based catalysts normally need to operate with >5000 turnovers and preferably at 10,000 turnovers or greater to be economically acceptable. In addition, these processes operate at high dilution, which gives unacceptable (and uneconomical) low reactor productivities. Further, these processes have not demonstrated effectiveness in generating the desired class of pivaloylacetate esters (1).

### SUMMARY OF THE INVENTION

The present invention relates to a novel and efficient process for generating pivaloylacetate esters via the carbonylation of chloropinacolone in the presence of a palladium catalyst. The process of the present invention converts chloropinacolone to the corresponding pivaloylacetate ester by contacting a chloropinacolone with carbon monoxide and an alcohol having the formula R¹OH, in the presence of a base and catalyst comprising a source of palladium and a coordinating, trisubstituted phosphine of formula (R³)₃P. In the foregoing description, R¹ and R³ are, independently, a C₁-C₁₀ alkyl, a C₃-C₁₀ cycloalkyl or a C₆-C₁₀ aryl group.

### DETAILED DESCRIPTION OF THE INVENTION

As stated above, the present invention is a process for converting chloropinacolone (2) to the corresponding pivaloylacetate ester (1) by contacting the chloropinacolone with carbon monoxide and an alcohol having the formula R¹OH, in the presence of a base and catalyst comprising a source of palladium and a coordinating, trisubstituted phosphine of formula (R³)₃P. The chemistry may be described as in Equation [1], below.

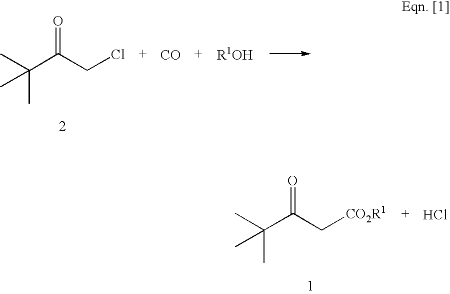

The chloropinacolone starting material is readily commercially available. Alternatively, it may be generated in high yield by chlorinating pinacolone, a process which is well known to those of skill in the art.

The alcohol for use in the present invention may be described by the general formula R¹OH, wherein R¹ is a C₁-C₁₀ alkyl, a C₃-C₁₀ cycloalkyl or a C₆-C₁₀ aryl group. Particular examples include methanol (R¹ is C₁ or methyl) or ethanol (R¹ is C₂ or ethyl). The molar ratio of R¹OH to chloropinacolone can range from about 1000:1 to about 1:1 of alcohol:chloropinacolone; the process is most productive when the ratio is between about 10:1 and about 1:1. The alcohol reactant for use herein may also function as a process solvent.

The catalyst for use in the present invention comprises a source of palladium and a source of an organic phosphine. The phosphine component, which may be described by the formula (R³)₃P, should be selected from phosphines wherein R³ is C₁-C₁₀ alkyl, C₃-C₁₀ cycloalkyl, or C₆-C₁₀ aryl. Each of the foregoing substituents for R³ may contain up to four heteroatoms, in addition to the carbon content, selected from S, N, or O. I have found high rates and high selectivity for the desired pivaloylacetate product, when R³ is cyclohexyl. Bis-chelating phosphines, such as diphosphines, such as 1,2-bis-(diphenylphosphino)-ethane and 1,3-bis-(diphenylphosphino)-propane, or tri-σ-toluyl phosphine, should not be used. In the Examples that follow, it is demonstrated that while heteroaromatic phosphines and triphenyl phosphines are useful in the process, the tricyclohexylphosphine based catalysts are superior performers.

The source of palladium for the catalyst is not critical and may be selected from any commercially available or readily generated palladium compound, salt, or complex. Common sources include palladium dihalides, such as palladium chloride; palladium carboxylate salts, such as palladium acetate; dibenzylideneacetone complexes of palladium (0); and nitrile complexes of palladium chloride. However, a particularly useful source of palladium is the preformed palladium phosphine complex, either as a chloride or acetate, the bis-tricyclohexylphosphinopalladium (II) dichloride complex being particularly convenient. The ratio of phosphine to palladium may vary over a wide range, with phosphine:Pd ratios of about 100:1 to about 1:1 being useful. Better results are obtained with phosphine:Pd ratios in the range of about 25:1 to about 2:1, with ratios in the range of about 15:1 to about 3:1 being particularly preferred.

The catalyst is operable over a wide range of chloropinacolone:Pd ratios, including ranges of about 50:1 to about 75,000:1. The choice of operating range for this ratio can help to optimize catalyst cost and reaction rates to improve overall economics. A preferred range of about 5,000:1 to about 25,000:1 should provide a good balance of catalyst cost and reaction rate, but this can vary as catalyst costs vary.

As shown in Equation [1], the reaction generates an equivalent of HCl. A base should be used to scavenge the HCl but can be deleterious to the selectivity and stability of the catalyst system. While the base may be selected from any base capable of neutralizing hydrogen halides, trialkyl amines of formula (R²)₃N are especially suitable for use herein, wherein R² is C₁-C₁₀ alkyl, C₃-C₁₀ cycloalkyl, or C₆-C₁₀ aryl. Preferred for use herein are trialkyl amines in which R² is an alkyl group of 2 to 4 carbons. Examples include triethyl amine, tripropyl amine, tributyl amine, and di-isopropyl ethyl amine. The ratio of base:chloropinacolone for use herein can be in the range of about 10:1 to about 1:1, with improved performance in the range of about 2:1 to about 1:1, and better performance in the range of about 1.25:1 to about 1.75:1. The foregoing trialkyl amine bases, and their salts, are readily soluble in the reaction medium, or can be readily dissolved upon warming or the addition of small amounts of alcohol, yet can be easily separated from the product mixture and recovered for further use by extraction and neutralization.

Although inorganic bases such as alkali and alkaline earth metal acetates, carbonates, alkoxides, and phosphates may be used, the lower solubility of such bases limits their usefulness because, for example, the high solids level hampers the ability to stir the reaction placing a mechanical constraint on the upper operable concentrations. By contrast, the relatively higher solubility of the trialkyl amines and their hydrohalide salts allows one to operate the reaction at higher concentrations than when operating with alkali and alkaline earth bases where.

In addition, trialkyl amines also demonstrate better selectivities toward the desired pivaloylacetates than when using the basic alkali or alkaline earth materials. While not being bound to any particular theory, it is believed that the foregoing effect of trialkyl amines may be traced to a balance of solubility, moderate basicity, and low nucleophilicity. Stronger bases seem to favor the formation of alkoxides resulting in an increased quantity of the alkoxypinacolone, and reduced quantities of the desired pivaloylacetate. This leads to yield losses and complicates separation work. Further, stronger bases can serve as nucleophiles and can generate additional substituted products, such as the acetoxypinacolone when acetate is used.

While the process may be operated at ambient pressures and temperatures, the rate of the reaction can be improved by operating at elevated temperature and carbon monoxide pressure. The process will operate at any temperature from about 0° C. to about 250° C.; however, reaction rate suffers at lower temperatures and selectivity suffers at elevated temperatures. Thus, the preferred temperature range is from about 75° C. to about 175° C., with the most preferred range being from about 100° C. to about 150° C.

The operable pressure for the reaction is in the range of about 1 to about 100 atmospheres (atm) absolute pressure (about 0.1 to about 10 MPa absolute pressure). Normally, the process is operated in the range of about 3 to about 50 atm (about 0.3 to about 5 MPa) absolute pressure. As the skilled artisan will appreciate, optimal pressure is a complex function of temperature and the nature and concentration of the reaction components, particularly the choice and concentration of alcohol and trialkyl amine, since these variables significantly affect the vapor pressure exerted by the reaction mixture. However, the preferred pressure range is about 5 to about 35 atm (about 0.5 to about 3.5 MPa) absolute pressure.

The skilled artisan will understand that each of the references herein to groups or moieties having a stated range of carbon atoms, such as “C₁-C₁₀-alkyl,” includes not only the C₁ group (methyl) and C₁₀ group (decyl) end points, but also each of the corresponding individual C₂, C₃, C₄, C₅ and so forth, groups. In addition, it will be understood that each of the individual points within a stated range of carbon atoms may be further combined to describe subranges that are inherently within the stated overall range. For example, the term “C₁-C₁₀-alkyl” includes not only the individual moieties C₁ through C₁₀, but also contemplates subranges such as “C₂-C₅-alkyl.”

This invention can be further illustrated by the following examples of preferred embodiments thereof, although it will be understood that these examples are included merely for purposes of illustration and are not intended to limit the scope of the invention.

### EXAMPLES

### Example 1

To a 300 mL Hastelloy-B autoclave was added 31.0 mL (24.5 g, 765 mmol) of methanol, 90.0 mL (70.0 g, 378 mmol) of tributyl amine, 33.0 mL (33.8 g, 251 mmol) of 1-chloropinacolone, 14.0 mg (0.019 mmol) of [(cyclohexyl)₃P)]₂PdCl₂, and 28.0 mg (0.1 mmol) of tricyclohexylphosphine. The autoclave was sealed, flushed with carbon monoxide, and pressurized to 2.0 atm. (0.2 MPa) gauge pressure with CO. The autoclave was then heated to 120° C. and the pressure was adjusted to 8.5 atm (0.86 MPa) gauge pressure. (The partial pressure of carbon monoxide is calculated to be 4.4 atm (0.45 MPa) after accounting for the vapor pressure of the reaction mixture.) The temperature and pressure were maintained using a continuous carbon monoxide feed for 3 h. The mixture was then cooled to yield a two phase reaction product. (The upper liquid layer is relatively small compared to the lower liquid layer.) The entire product mixture (both layers) was diluted with 50.0 mL (39.55 g) of methanol to generate a homogeneous mixture and was then analyzed for pinacolone (an undesired by-product of the reaction), chloropinacolone and methyl pivaloylacetate using gas chromatography. The analysis indicated there was 0.47 wt. % pinacolone, 2.24 wt. % chloropinacolone, and 17.82 wt. % methyl pivaloylacetate (including the methanol added to make the mixture homogeneous.) This represents a conversion of 89% with a selectivity of 88% toward the desired methyl pivaloylacetate and 3.7% selectivity toward the undesired pinacolone where conversion and selectivity are defined by the equations:

$$Conversion = \frac{{mol}_{CIP added} - {mol}_{CIP product}}{{mol}_{CIP added}} \times 100 \%$$

$${Selectivity}_{i} = \frac{{mol}_{i}}{{mol}_{CIP added} - {mol}_{CIP product}} \times 100 \%$$

where mol_{CIP added}=moles chloropinacolone added,
   mol_{CIP product}=moles chloropinacolone in product,
   i=methyl pivaloylacetate or pinacolone,
and molar quantities are determined by the equations:

$${mol}_{mpa} = \frac{{x}_{mpa} {wt}_{0}}{[ {MW}_{mpa} - ( {x}_{mpa} * {MW}_{co} ) ]}$$

$${mol}_{CIP product} = \frac{{x}_{CIP} [ {wt}_{0} + ( {mol}_{mpa} * {MW}_{co} ) ]}{{MW}_{CIP product}}$$

$${mol}_{P} = \frac{{x}_{P} [ {wt}_{0} + ( {mol}_{mpa} * {MW}_{co} ) ]}{{MW}_{P}}$$
where
   molₘₚₐ=moles of methyl pivaloylacetate in product
   mol_{CIP product}=moles of methyl pivaloylacetate in product
   xₘₚₐ=weight fraction of methyl pivaloyl acetate (=wt. % methyl pivaloylacetate by GC/100)
   x_{CIP}=weight fraction of chloropinacolone (=wt. % chloropinacolone by GC/100)
   x_{P}=weight fraction of pinacolone (=wt. % pinacolone by GC/100)
   MWₘₚₐ=molecular weight of methyl pivaloylacetate (158.2 g/mol)
   MW_{co}=molecular weight of CO (28.0 grams/mol),
   MW_{CIP}=molecular weight of chloropinacolone (134.6 g/mol)
   MW_{P}=molecular weight of pinacolone (100.2 g/mol), and
   wt₀=weight of initial solution+50 grams MeOH used for homogenizing solution (178.3 grams).
      The turnover number (TON) was 10,280 mol methyl pivaloylacetate/mol Pd, where the turnover number is determined by the equation:
      TON=mol_{mpa formed}/mol_{Pd added}

The results also appear in tables 1 and 2.

### Example 2-7

Example 1 was repeated except that the pressure and temperature were changed as indicated in tables 1 and 2. Results appear in Table 1, which summarizes the gas chromatographic analyses, and Table 2, which summarizes the conversion, selectivity, and turnover numbers which are calculated from the gas chromatographic analyses as indicated above. Results appear in tables 1 and 2.

### Examples 8-10

Example 2 was repeated using differing amounts of [(cyclohexyl)₃P)]₂PdCl₂, and tricyclohexylphosphine catalyst as indicated in tables 1 and 2. Results appear in tables 1 and 2.

### Examples 11-14

Example 1 was repeated except the amount of phosphine used was varied as indicated in tables 1 and 2. Results appear in tables 1 and 2.

TABLE 1
Summary of Data for Examples 1-14.

| | Pd | Additional | | | Gauge | | | |
|---|---|---|---|---|---|---|---|---|
| Ex. | Catal.¹ | Cy-hexyl₃P | P/Pd | Temp. | Press | GC Analysis (wt. %) | | |
| No. | (mmol) | (mmol) | ratio | (° C.) | (atm) | Pinacolone | ChloroPinacolone | MPA² |
| 1 | 0.019 | 0.1 | 7.26 | 120 | 8.5 | 0.47 | 2.24 | 17.82 |
| 2 | 0.019 | 0.1 | 7.26 | 120 | 5.4 | 0.37 | 4.25 | 16.43 |
| 3 | 0.019 | 0.1 | 7.26 | 120 | 17.0 | 0.26 | 6.22 | 13.89 |
| 4 | 0.019 | 0.1 | 7.26 | 120 | 34.0 | 0.18 | 8.47 | 8.07 |
| 5 | 0.019 | 0.1 | 7.26 | 130 | 17.0 | 0.38 | 4.35 | 14.68 |
| 6 | 0.019 | 0.1 | 7.26 | 140 | 17.0 | 1.20 | 0.53 | 17.92 |
| 7 | 0.019 | 0.1 | 7.26 | 150 | 17.0 | 3.45 | 0.23 | 13.43 |
| 8 | 0.1 | 0.2 | 4 | 120 | 5.4 | 0.43 | 0.26 | 19.34 |
| 9 | 0.05 | 0.2 | 6 | 120 | 5.4 | 0.24 | 6.64 | 13.91 |
| 10 | 0.0095 | 0.05 | 7.26 | 120 | 5.4 | 0.18 | 10.49 | 8.48 |
| 11 | 0.019 | 0 | 2 | 120 | 8.5 | 0.20 | 5.67 | 11.84 |
| 12 | 0.019 | 0.05 | 4.63 | 120 | 8.5 | 0.27 | 3.08 | 15.28 |
| 13 | 0.019 | 0.15 | 9.89 | 120 | 8.5 | 0.31 | 4.46 | 15.70 |
| 14 | 0.019 | 0.2 | 12.5 | 120 | 8.5 | 0.26 | 5.06 | 15.61 |

¹Palladium catalyst = [(cyclohexyl)₃P)]₂PdCl₂
²MPA = methyl pivaloylacetate

TABLE 2
Summary of Conversion, Selectivity, and Turnover Numbers
(TON) for Examples 1-14.

| | | Additional | | | | | | | |
|---|---|---|---|---|---|---|---|---|---|
| | Pd | Cy- | | | Gauge | | Selectivity | | TON |
| Ex. | Catal.¹ | hexyl₃P | P/Pd | Temp. | Press | Conversion | (%) | | (mol pdt./ |
| No. | (mmol) | (mmol) | ratio | (° C.) | (atm) | (%) | MPA² | Pinacolone | mol Pd) |
| 1 | 0.019 | 0.1 | 7.26 | 120 | 8.5 | 89 | 88 | 3.7 | 10280 |
| 2 | 0.019 | 0.1 | 7.26 | 120 | 5.4 | 78 | 91 | 3.2 | 9450 |
| 3 | 0.019 | 0.1 | 7.26 | 120 | 17.0 | 68 | 88 | 2.6 | 7960 |
| 4 | 0.019 | 0.1 | 7.26 | 120 | 34.0 | 57 | 60 | 2.1 | 4570 |
| 5 | 0.019 | 0.1 | 7.26 | 130 | 17.0 | 78 | 82 | 3.3 | 8420 |
| 6 | 0.019 | 0.1 | 7.26 | 140 | 17.0 | 97 | 80 | 8.5 | 10340 |
| 7 | 0.019 | 0.1 | 7.26 | 150 | 17.0 | 99 | 59 | 23.9 | 7690 |
| 8 | 0.1 | 0.2 | 4 | 120 | 5.4 | 99 | 86 | 3.0 | 2130 |
| 9 | 0.05 | 0.2 | 6 | 120 | 5.4 | 66 | 91 | 2.5 | 3030 |
| 10 | 0.0095 | 0.05 | 7.26 | 120 | 5.4 | 47 | 77 | 2.6 | 9620 |
| 11 | 0.019 | 0 | 2 | 120 | 8.5 | 71 | 72 | 1.9 | 6760 |
| 12 | 0.019 | 0.05 | 4.63 | 120 | 8.5 | 84 | 79 | 2.2 | 8770 |
| 13 | 0.019 | 0.15 | 9.89 | 120 | 8.5 | 77 | 88 | 2.8 | 9020 |
| 14 | 0.019 | 0.2 | 12.5 | 120 | 8.5 | 74 | 91 | 2.4 | 8970 |

¹Palladium catalyst = [(cyclohexyl)₃P)]₂PdCl₂
²MPA = methyl pivaloylacetate

### Example 15

To a 300 mL Hastelloy-B autoclave was added 31.0 mL (24.5 g, 765 mmol) of methanol, 72.0 mL (52.4 g, 378 mmol) of tripropyl amine, 33.0 mL (33.8 g, 251 mmol) of 1-chloropinacolone, 14.0 mg (0.019 mmol) of [(cyclohexyl)₃P)]₂PdCl₂, and 28.0 mg (0.1 mmol) of tricyclohexylphosphine. The autoclave was sealed, flushed with carbon monoxide, and pressurized to 2.0 atm. (0.2 MPa) gauge pressure with CO. The autoclave was then heated to 120° C. and the pressure was adjusted to 5.4 atm (0.55 MPa) gauge pressure. The temperature and pressure were maintained using a continuous carbon monoxide feed for 3 h. The mixture was then cooled to yield a two phase reaction product. (The upper liquid layer is relatively small compared to the lower liquid layer.) The entire product mixture (both layers) was diluted with 50.0 mL (39.55 g) of methanol to generate a homogeneous mixture and was then analyzed for pinacolone (an undesired by-product of the reaction), chloropinacolone and methyl pivaloylacetate using gas chromatography. The analysis indicated there was 0.25 wt. % pinacolone, 7.58 wt. % chloropinacolone, and 13.71 wt. % methyl pivaloylacetate (including the methanol added to make the mixture homogeneous.) This represents a conversion of 65% with a selectivity of 65% toward the desired methyl pivaloylacetate and 2.4% selectivity toward the undesired pinacolone where conversion and selectivity are calculated as described in example 1. The turnover number was 7110 moles methyl pivaloylacetate/mole Pd.

### Example 16

To a 300 mL Hastelloy-B autoclave was added 31.0 mL (24.5 g, 765 mmol) of methanol, 53.0 mL (38.5 g, 380 mmol) of tributyl amine, 33.0 mL (33.8 g, 251 mmol) of 1-chloropinacolone, 14.0 mg (0.019 mmol) of [(cyclohexyl)₃P)]₂PdCl₂, and 28.0 mg (0.1 mmol) of tricyclohexylphosphine. The autoclave was sealed, flushed with carbon monoxide, and pressurized to 2.0 atm. (0.2 MPa) gauge pressure with CO. The autoclave was then heated to 120° C. and the pressure was adjusted to 9.5 atm (0.96 MPa) gauge pressure. The temperature and pressure were maintained using a continuous carbon monoxide feed for 3 h. The mixture was then cooled to yield a mixture of a liquid phase and precipitated solids. The entire product mixture (both the solids and liquids were dissolved in 100 mL (79.1 g) of methanol to generate a homogeneous mixture and was then analyzed for pinacolone (an undesired by-product of the reaction), chloropinacolone and methyl pivaloylacetate using gas chromatography. The analysis indicated there was 0.26 wt. % pinacolone, 2.04 wt. % chloropinacolone, and 12.06 wt. % methyl pivaloylacetate (including the methanol added to make the mixture homogeneous.) This represents a conversion of 88% with a selectivity of 68% toward the desired methyl pivaloylacetate and 2.3% selectivity toward the undesired pinacolone where conversion and selectivity are calculated as described in Example 1. The turnover number was 8020 moles methyl pivaloylacetate/mole Pd.

### Example 17

To a 300 mL Hastelloy-B autoclave was added 110 mL (87.0 g, 2.71 mol) of methanol, 36.0 mL (28.0 g, 151 mmol) of tributyl amine, 13.1 mL (13.4 g, 100 mmol) of 1-chloropinacolone, and 73.8 mg (0.1 mmol) of [(cyclohexyl)₃P)]₂PdCl₂. The autoclave was sealed, flushed with carbon monoxide, and pressurized to 2.0 atm. (0.2 MPa) with CO. The autoclave was then heated to 120° C. and the pressure was adjusted to 10.2 atm. (1.03 MPa). The temperature and pressure were maintained using a continuous carbon monoxide feed for 3 h. The mixture was then cooled and analyzed by gas chromatography which indicated that the reaction product contained 0.15 wt. % pinacolone, 0.17 wt. % chloropinacolone, and 10.86 wt. % methyl pivaloylacetate. This corresponds to a 98% conversion with a selectivity of 92% for the desired methyl pivaloylacetate and 2.0% toward the undesired pinacolone. (Calculated as in Example 1.) The turnover number was 899 mole methyl pivaloylacetate/mole Pd.

### Example 18

Example 17 was repeated except that 70.2 mg (0.1 mmol) of (Ph₃P)₂PdCl₂ was substituted for the [(cyclohexyl)₃P)]₂PdCl₂. Gas chromatographic analysis indicated that the reaction product contained 0.66 wt. % pinacolone, 3.03 wt. % chloropinacolone, and 5.72 wt. % methyl pivaloylacetate. This corresponds to a 71% conversion with a selectivity of 67% for the desired methyl pivaloylacetate and 12.1% toward the undesired pinacolone. (Calculated as in Example 1.) The turnover number was 469 mole methyl pivaloylacetate/mole Pd.

### Example 19

To a 300 mL Hastelloy-B autoclave was added 110 mL (87.0 g, 2.71 mol) of methanol, 30.0 mL (23.3 g, 126 mmol) of tributyl amine, 11.0 mL (11.3 g, 83.8 mmol) of 1-chloropinacolone, and 73.8 mg (0.1 mmol) of [(cyclohexyl)₃P)]₂PdCl₂. The autoclave was sealed, flushed with carbon monoxide, and pressurized to 2.0 atm (0.2 MPa) with CO. The autoclave was then heated to 105° C. and the pressure was adjusted to 5.4 atm (0.55 MPa). The temperature and pressure were maintained using a continuous carbon monoxide feed for 3 h. The mixture was then cooled and analyzed by gas chromatography which indicated that the reaction product contained 0.10 wt. % pinacolone, 1.63 wt. % chloropinacolone, and 8.40 wt. % methyl pivaloylacetate. This corresponds to an 82% conversion with a selectivity of 95% for the desired methyl pivaloylacetate and 1.8% toward the undesired pinacolone. (Calculated as in Example 1.) The turnover number was 656 mole methyl pivaloylacetate/mole Pd.

### Example 20

Example 19 was repeated except that 70.2 mg (0.1 mmol) of (Ph₃P)₂PdCl₂ was substituted for the [(cyclohexyl)₃P)]₂PdCl₂. Gas chromatographic analysis indicated that the reaction product contained 0.33 wt. % pinacolone, 3.77 wt. % chloropinacolone, and 5.37 wt. % methyl pivaloylacetate. This corresponds to a 59% conversion with a selectivity of 84% for the desired methyl pivaloylacetate and 8.2% toward the undesired pinacolone. (Calculated as in Example 1.) The turnover number was 417 mole methyl pivaloylacetate/mole Pd.

### Example 21

Example 19 was repeated except that 17.7 mg (0.1 mmol) of PdCl₂ and 52.7 mg (0.2 mmol) of (2-pyridyl)diphenyl phosphine was substituted for the [(cyclohexyl)₃P)]₂PdCl₂. Gas chromatographic analysis indicated that the reaction product contained 0.16 wt. % pinacolone, 4.09 wt. % chloropinacolone, and 4.87 wt. % methyl pivaloylacetate. This corresponds to a 55% conversion with a selectivity of 81% for the desired methyl pivaloylacetate and 4.2% toward the undesired pinacolone. (Calculated as in Example 1.) The turnover number was 378 mole methyl pivaloylacetate/mole Pd.

### Example 22

Example 19 was repeated except that 17.7 mg (0.1 mmol) of PdCl₂ and 42.7 mg (0.1 mmol) of 1,4-bis-(diphenylphosphino)-butane was substituted for the [(cyclohexyl)₃P)]₂PdCl₂. Gas chromatographic analysis indicated that the reaction product contained 0.32 wt. % pinacolone, 6.67 wt. % chloropinacolone, and 0.24 wt. % methyl pivaloylacetate. This corresponds to a 28% conversion with a selectivity of 8% for the desired methyl pivaloylacetate and 16.6% toward the undesired pinacolone. (Calculated as in Example 1.) The turnover number was 18 mole methyl pivaloylacetate/mole Pd. This example demonstrates that bidentate ligand, exemplified by the bridging phosphine, 1,4-bis-(diphenylphosphino)-butane, while capable of producing small amounts of the desired methyl pivaloylacetate, give markedly poorer performance with respect to rate and selectivity.

The invention has been described in detail with particular reference to preferred embodiments thereof, but it will be understood that variations and modifications can be effected within the spirit and scope of the invention.

1. A process for producing pivaloylacetate esters of formula (1) 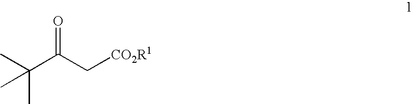
which comprises contacting chloropinacolone (2) 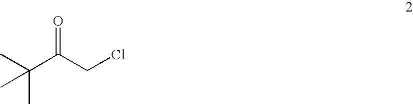
with carbon monoxide and an alcohol of formula R¹OH in the presence of a base and a catalyst comprising palladium and a trisubstituted phosphine wherein R¹ is C₁-C₁₀ alkyl, C₃-C₁₀ cycloalkyl or C₆-C₁₀ aryl.

2. A process according to claim 1 wherein the base is represented by (R²)₃N and the phosphine is represented by (R³)₃P and wherein R¹, R² and R³ are, independently, C₁-C₁₀ alkyl, C₃-C₁₀ cycloalkyl or C₆-C₁₀ aryl.

3. A process according to claim 2 wherein R¹ is methyl or ethyl; the base is triethyl amine, tripropyl amine, tributyl amine, or di-isopropyl ethyl amine; and the phosphine is tricyclohexylphosphine.

4. A process according to claim 3 wherein R¹ is methyl and the base is triethyl amine.

5. A process according to claim 1 wherein the contacting is performed at a temperature of about 75° C. to about 175° C. and at a pressure of about 3 to about 50 atm.

6. A process according to claim 5 wherein the contacting is performed at a temperature of about 100° C. to about 150° C. and at a pressure of about 5 to about 35 atm.

7. A process for producing pivaloylacetate esters of formula (1) 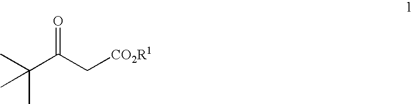
which comprises contacting chloropinacolone (2) 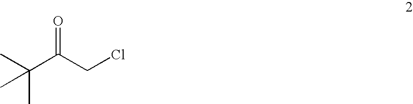
with carbon monoxide and an alcohol represented by R¹OH in the presence of a base having formula (R²)₃N and a catalyst comprising palladium and a trisubstituted phosphine having formula (R³)₃P, wherein R¹, R² and R³ are, independently, C₁-C₁₀ alkyl, C₃-C₁₀ cycloalkyl or C₆-C₁₀ aryl and the contacting is performed at a temperature of about 75° C. to about 175° C. and at a pressure of about 3 to about 50 atm.

8. A process according to claim 7 wherein R¹ is methyl or ethyl; the base is triethyl amine, tripropyl amine, tributyl amine, or di-isopropyl ethyl amine; the phosphine is tricyclohexylphosphine; and the contacting is performed at a temperature of about 100° C. to about 150° C. and at a pressure of about 5 to about 35 atm.

9. A process according to claim 8 wherein R¹ is methyl and the base is triethyl amine.

10. A process for producing pivaloylacetate esters of formula (1) 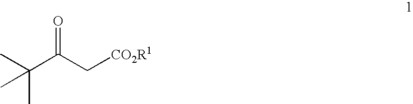
which comprises contacting chloropinacolone (2) 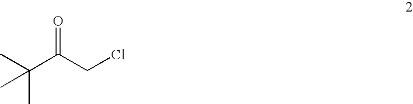
with carbon monoxide and an alcohol represented by R¹OH in the presence of a base and a catalyst comprising palladium and tricyclohexylphosphine, wherein R¹ is methyl or ethyl, the base is triethyl amine, tripropyl amine, tributyl amine, or di-isopropyl ethyl amine, and the contacting is performed at a temperature of about 75° C. to about 175° C. and at a pressure of about 3 to about 50 atm.

11. A process according to claim 10 wherein R¹ is methyl and the base is triethyl amine.

12. A process according to claim 11 wherein the contacting is performed at a temperature of about 100° C. to about 150° C. and at a pressure of about 5 to about 35 atm.
